(19) United States
(12) Patent Application Publication
(10) Pub. No.: US 2008/0027416 A1
(43) Pub. Date: Jan. 31, 2008

(54) Catheter with Light Source

(76) Inventors: Kory P. Hamel, Bloomington, MN (US); Vincent G. Copa, Minnetonka, MN (US); Adam L. Gullickson, Richfield, MN (US)

Correspondence Address: AMS RESEARCH CORPORATION, 10700 BREN ROAD WEST, MINNETONKA, MN, 55343, US

(21) Appl. No.: 11/828,208

(22) Filed: Jul. 25, 2007

Related U.S. Application Data
(60) Provisional application No. 60/820,244, filed on Jul. 25, 2006.

(51) Int. Cl. A61M 27/00; A61B 17/11

(52) U.S. Cl. 604/544; 606/153

(57) ABSTRACT

An improved catheter design and related methods providing for the illumination of various anatomical structures while placed within a lumen. The catheter can include a drainage conduit, hose or tube that extends from a proximal end connected to a collection bag to a distal end with a catheter head or opening for collecting fluid. An expandable retention balloon is disposed near the distal end and is adapted to expand within the confines of the bladder by injecting a gas or sterile liquid under pressure through a filling tube or channel disposed axially within the catheter. At least one illumination source, such as a Light Emitting Diode (LED) or a fiber optic light source, can be disposed proximate the catheter head. The control of the light source can include a separate channel disposed axially in the catheter from the proximal end to the distal end.

### PRIORITY CLAIM

The present application claims priority to U.S. Provisional Application No. 60/820,244 filed Jul. 25, 2006 and entitled, “CATHETER WITH LIGHT SOURCE”, which is herein incorporated by reference in its entirety.

### FIELD OF THE INVENTION

The invention relates to catheters and procedures related to catheter placement, and particularly to an improved means of illuminating and visualizing the bladder and nearby structure during a catheterization procedure.

### BACKGROUND OF THE INVENTION

A catheter is a tubular medical device for insertion into canals, vessels, passageways or body cavity as an access means for introduction or removal of fluids or as a spacer to maintain the shape of the passage. One typical use of catheters is to permit urinary bladder drainage in certain patients who have had major surgery and/or trauma, or in any patient unable to urinate. However, there are some serious drawbacks to the standard indwelling urinary catheters now available. The widely-used Foley catheter is inserted in the urethra and up to the bladder where a retention balloon is inflated. As the most sensitive areas of the bladder are the trigone and bladder neck areas where the balloon rests, placement of the balloon is critical. In rare instances, the balloon may be inadvertently inflated inside of the urethra, causing trauma to the urethra and resulting thereby in pain, bleeding and possible stricture formation within the urethra. Thus there is a need to insure proper positioning of the catheter.

Catheters are also used for treatment of the prostate and bladder. The catheter promotes healing after surgery as it is important to drain the bladder by an indwelling urinary catheter. In addition, thousands of prostatectomies are performed each year involving the use of a catheter. For example, in a radical prostatectomy, the surgeon removes all or most of the patient's prostate. Because the urethra travels through the prostate immediately before reaching the bladder, the upper part of the urethra is removed in the surgery. The procedure leaves a severed urethral stump and a severed bladder neck.

To restore proper urinary functions, the bladder and the urethra must be reconnected by a laparoscopic technique known as an urethrovesical anastomosis. Anastomosis is the union or joinder of one hollow vessel or structure to another so that the interior of the vessels communicate with one another. Conventionally, a surgeon can execute delicate suturing operations with tiny, fine needles to reconnect these anatomical bodies. Installation of sutures, however, with a needle, to connect the severed tissues, can be a difficult and often a technique-sensitive task. Many factors can make this task difficult, including a very small amount of tissue to work with (at the urethral stump and at the bladder neck), proximal urethras at the bladder, and a proximal nerve bundle and sphincter at the urethral stump. All of these add up to a complicated and delicate suturing procedure that, if not performed properly, could result in complications such as leakage, difficulty in healing or failure to heal, incontinence, or impotence. Specific problems include necrosis of the sutured tissues; stricture of the urethra, which impedes the flow of fluid through it; and a urethra-bladder connection that is not fluid-tight. In addition, methods of suturing the urethra to the bladder allow for accidental or inadvertent piercing of the nearby neurovascular bundle, which can cause incontinence or impotence.

In one method a catheter body with a tissue approximation structure as defined in U.S. Pat. App. Pub. 2004/0087995 to Vincent G. Copa, et. al, is inserted into the bladder. The catheter allows for drainage of the bladder during the healing process while the tissue approximation structure causes contact between the severed tissues so as to maintain contact during healing. Specifically, there is a need to improve the technique used to connect the urethra to the bladder neck to perform an urethrovesical anastomosis, e.g., after removal of the prostate. Thus it is clear there is an unmet need for an improved apparatus and technique for directly visualizing the bladder neck/prostate interface for catheter placement or for bladder neck dissection.

### SUMMARY OF THE INVENTION

The present invention is a catheter with a light source for improving visual identification of the bladder neck for relative placement of a catheter or for bladder neck dissection. The improved catheter includes a light source built into the lumen, the balloon or the fixation section of the catheter. The light sources can be a fiber optic device, an LED, an illumination solution pumped into a catheter balloon or a combination of multiple sources. The balloon can be constructed of a material conducive to the transmission of the light source, including transparent, translucent, or reflective material.

In a first example, the present invention is intended to improve positioning of the catheter relative to the bladder neck for bladder drainage. Any catheter which is inserted into the bladder and allowed to remain in the bladder is called an indwelling catheter. A common type of indwelling catheter is a Foley catheter. A Foley catheter has a balloon attachment at one end. After the Foley catheter is inserted into the urethra, the balloon is filled with sterile water. The filled balloon prevents the catheter from leaving the bladder. By illuminating the drainage end of the Foley catheter the placement of the balloon can be set inside the bladder relative to the bladder neck. The base of the balloon is thus illuminated by the present invention by including an illumination mechanism with the balloon itself or to the catheter body upstream of the balloon. The illumination mechanism can be within the balloon through the inclusion of a fiber optic or LED. The balloon can also be filled with a sterile liquid having fluorescent properties.

In another embodiment, the catheter of the present invention is used for surgical procedures involving the prostate or the bladder. It is envisioned that a catheter, such as a Foley catheter, would be inserted into the urethra. The distal end of the catheter would be inserted into the bladder while the catheter can include tissue approximating structure to assist with a surgical procedure. The illumination system can be placed relative to the tissue approximating structure so that the surgeon can better visualize the procedure. The illuminations system can involve a fiber optic light source or an LED or both.

It is further envisioned that alternate embodiments can include illumination sources in multiple locations along the length of the catheter. For example, a Foley catheter can include an illumination source within the balloon and an LED or fiber optic source proximate a tissue approximating structure. The balloon illumination can assist with seating the catheter within the bladder while the second source helps the surgeon visualize a prostatectomy for example.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follows more particularly exemplify these embodiments.

### BRIEF DESCRIPTION OF THE DRAWINGS

These as well as other objects and advantages of this invention, will be more completely understood and appreciated by referring to the following more detailed description of the presently preferred exemplary embodiments of the invention in conjunction with the accompanying drawings of which:

FIG. 1 is a perspective view of a Foley catheter including the present invention.

FIG. 2 is a cross sectional view of a Foley catheter with the present invention disposed within a bladder.

FIG. 3 is a cross sectional view of the catheter body of the Foley catheter.

FIG. 4 is a perspective view of an alternative embodiment of an anastomosis device of the present invention.

### DETAILED DESCRIPTION OF THE DRAWINGS

As required, detailed embodiments of the present invention are disclosed herein, however, it is to be understood that the disclosed embodiments are merely exemplary of the invention which can embodied in various forms. Therefore, specific structural and functional details disclosed herein are not to be interpreted as limiting, but merely as a basis for teaching one skilled in the art to variously employ the present invention in virtually any appropriately detailed construction.

The present invention is an improved catheter design that allows for the illumination of the various anatomical structures while placed within a lumen. An example of a useful design, generally, is the type sometimes referred to as a Foley catheter that has been constructed to include modified features as also presented in the present description including tissue approximating structure. In general, the catheter in question includes a drainage conduit, hose or tube that extends from a proximal end connected to a collection bag to a distal end with a catheter head or opening for collecting fluid. In one example the distal end is inserted into the bladder by way of the urethra. An expandable retention balloon is disposed near the distal end and is adapted to expand within the confines of the bladder by inserting into the balloon a gas or sterile liquid under pressure through a filling tube or channel disposed axially within the catheter. At least one illumination source, such as a Light Emitting Diode (LED) or a fiber optic light source, can be disposed proximate the catheter head. The control of the light source can include a separate channel disposed axially in the catheter from the proximal end to the distal end. Additional illumination sources can require independent control channels so along or internal to the catheter body.

FIG. 1 shows a single example of a modified-Foley-catheter-type with illumination device according to the invention. Device 10 includes catheter body 12 having a distal end 11 and opposing proximal end 15. Device 10 includes balloon 13, illumination device 16, and drainage aperture 14 at proximal end 15. A typical Foley catheter can include a drainage lumen 17 and an inflation lumen 18 for inflating and deflating the balloon 13. The balloon 13 is normally deflated until properly positioned in a patient's bladder. Once the catheter 10 is properly positioned, the inflation lumen 18 delivers fluid or air under pressure to inflate the balloon 13.

Still referring to FIG. 1, device 10 includes at proximal end 15 a port 20 that can be connected to attachment 21. Such proximal end attachment configurations are well known, and such known or future developed proximal ends and attachments will be understood to be useful according to devices and methods of the invention. In the illustrated embodiment, proximal end 15 also includes a port 19 that can connect to a lumen (not shown) such as an illumination lumen for illumination device 16. Likewise, port 20 allows for passage of lumens for drainage lumen from aperture 17. Another port, 18, part of attachment 21, can also be used with an inflation lumen or a drainage lumen.

FIG. 2 is an illustration of the present invention 10 disposed within bladder 22. The catheter 10 is inserted into the urethra 23 with distal end 11 extending through prostate 24 into bladder 22. Balloon 13 is inflated so that balloon base 25 rests proximate the bladder neck 26. Drainage aperture 14 is thus positioned within the bladder 22 to affect drainage. Illumination device 16 is depicted in this embodiment as a radial ring of LEDs situated at bladder neck 26. Illumination device 16 could also be one or more fiber optic light sources. Alternative placement of illumination device 16 could be within balloon 13 or a proximate drainage aperture 14.

Control or activation of illumination source 16 is provided by an appropriate connection within the catheter body as depicted in FIG. 3. Drainage lumen 27 extends axially within catheter 10 along with inflation lumen 28 and illumination control lumen 29. If multiple illumination sources are provided, for example one ring at the bladder neck 26 and one proximate the drainage aperture 14, multiple illumination lumens can be required as well. Illumination lumen 29 can further contain a fiber optic strand for a fiber optic light source and/or a conductor for the LED control.

In an alternative embodiment, the present invention can be used in treatment of prostate disorders or cancers. An anastomosis device of the present invention typically includes a hollow, elongate, flexible catheter body having a proximal end and a distal end with at least one illumination device near the distal end. An inflatable balloon can be located near the distal end, and an inflation lumen for inflating the balloon can extend to the balloon along the catheter body, e.g., along a portion or all of the catheter body from the proximal end to the balloon. The balloon section can include an illumination source as described in FIGS. 1-3.

The illumination device provides a number of benefits. During use, the illumination device provides means to visualize the proper positioning of the balloon in the bladder. The balloon should rest against the neck of the bladder to prevent urine from entering the neck and to prevent urine from contacting the anastomosis site. Urine at the anastomosis site has the potential to cause difficulties in healing or to cause a stricture, among other deleterious effects. With the balloon blocking the bladder neck during use, urine will pool in the bladder and can be drained from the bladder, for example, using one or more draining apertures at the distal end of the anastomosis device connected to a drainage lumen. A drainage lumen can extend from one or more drainage apertures near the distal end, e.g., from apertures at the distal end to a location that is at or near the proximal end. As a particular example, a port can be present at the proximal end to connect the drainage lumen to a urine collection device.

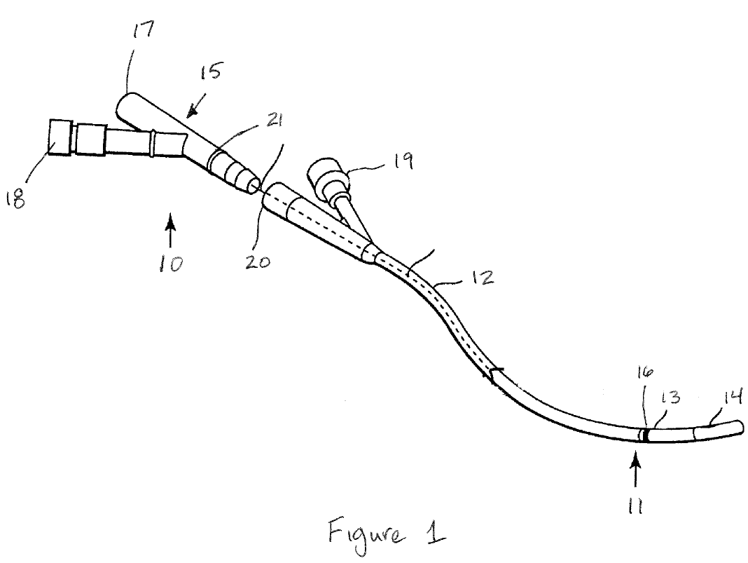

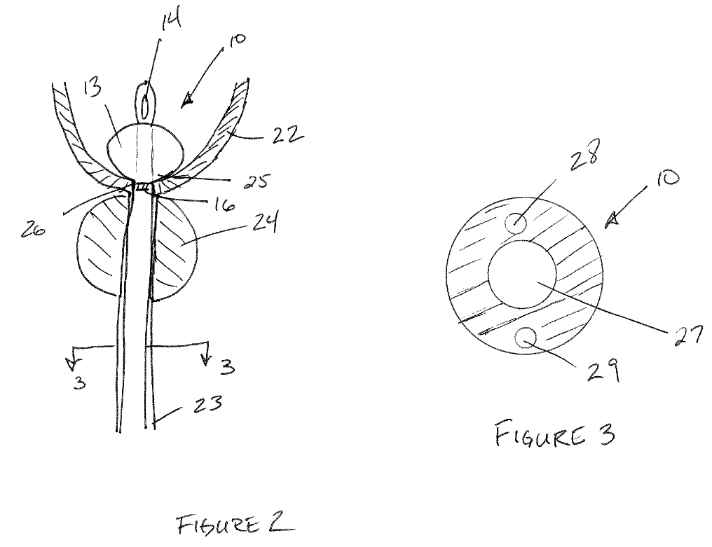

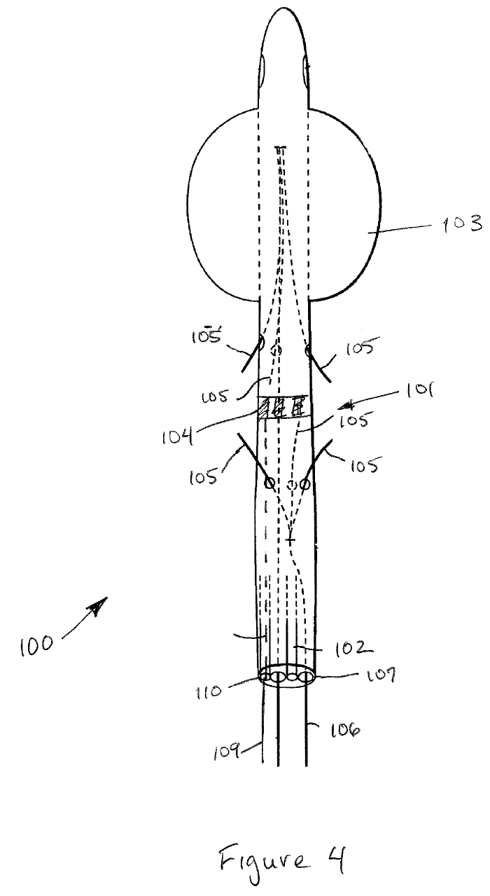

The illumination device can also be advantageous in the positioning of the tissue approximating structure relative to the bladder neck. In an anastomosis device 100, tissue approximating structure 101 can be located along the catheter body 102, for example, as illustrated in FIG. 4, along catheter body 102 approximately midway between the far ends of the device. This location can be useful, for example, in performing an end-to-end urethral anastomosis procedure; a location closer to balloon 103 can be useful for a vesico-urethral anastomosis. Illumination device 104 can be disposed on the catheter body cephalad or caudad in relation to the tissue approximating structure 101. One alternative would be to dispose illuminating devices on to the tissue approximating structures (e.g., the tines 105). Another alternative would be to illuminate the balloon itself as light from the balloon would be seen through the bladder wall.

Tissue approximating structure 101 of device 100 is shown as a single set of tines 105 (shown in an extended position) but can alternatively include an additional set of (optionally opposing) tines, a different type of elongate structure such as a probe or prod or needle, a balloon, or any other structure that can be used to place or hold severed tissue in contact with another opposing severed tissue for healing. The tissue approximating structure 101 can be actuated by any useful method or device or structure, for example as illustrated in the attached figures, by an elongate actuator mechanism 106 extending through a lumen 107 in a catheter body 102. The actuator mechanism 106 can be of the same material as the tissue approximating structure 101, or can be a different material secured to the tissue approximating structure 101. The actuator 106 can extend out of the proximal end of a device 108 or can enter the device through a lateral port located at the proximal end of the device (not shown). Likewise, illumination device 104 can be actuated by a conductor 109 extending through a lumen 110 in catheter body 102. For a fiber optic light source the conductor 109 would be substituted with a fiber optic strand disposed within lumen 110.

Overall, an anastomosis device can contain various lumens (e.g., for inflating a balloon, for drainage, for containing actuating mechanisms for tissue approximating structure, for activation of an illumination device, etc.) and actuating mechanisms running along at least a partial length of a catheter body. The mechanisms and lumens can be arranged in any useful configuration such as coaxially, side-by-side, or according to any other useful configuration. A lumen or a mechanism (e.g., actuating mechanism) that runs along at least a portion of the length of the catheter body can be diverted at the proximal end of the catheter body to a port that provides access to the lumen or mechanism during use, as is known.

The catheter and its componentry, including the illumination devices, can be made of materials normally used and known to be useful for such devices, or future developed materials, especially including known or future developed materials that are relatively inert and biocompatible. For example, a catheter body can be prepared from a flexible plastic or polymeric material. Examples of presently understood materials that can be useful for a catheter body can include silicones, latex, rubbers, polyurethanes, and combinations of these or other materials.

Alternate embodiments of illumination sources attached to catheters and anastomosis devices (and related methods) will be useful according to this description, as will be appreciated by those of skill, even if not specifically illustrated or described. For example, combinations of the above described features of an anastomosis device, as well as other features such as additional or different structures or protrusions, can be useful to function as tissue approximating structures. Examples can include a second balloon or other movable, inflatable, or stationary structures. Additionally, the number or illumination devices and their relative location and number along the catheter body can be of any useful variation, as can be the mechanism by which the illumination device is actuated.

What is claimed is:

1. A medical device probe comprising;
a hollow elongate flexible catheter body having a proximal and a distal end,
an inflatable balloon at the distal end,
a drainage lumen connected to the distal end, and
an illumination system disposed proximate the inflatable balloon, the illumination system including an illumination lumen having a proximal end and a distal end disposed within the catheter body, an actuating mechanism proximate the distal end operatively connected to at least one illumination device, the illumination device disposed proximate the distal end of the illumination lumen.

2. The medical device probe of claim 1 further including a tissue approximating structure connected to the catheter body, said tissue approximation structure disposed on the proximal side of the balloon at a location to contact severed tissue during an anastomosis technique.

3. The medical device probe of claim 2 wherein the tissue approximating structure comprises moveable elongate structures selected from a tine, a probe, a prod and a needle.

4. The medical device probe of claim 3 wherein the tissue approximating structure can be extended and retracted from at least one aperture in the catheter body using an actuating mechanism that extends through a lumen extending axially through the catheter body.

5. The medical device probe of claim 4 wherein the illumination device is mounted on the elongate structures, the illumination device operatively connected by a conductor to the actuating mechanism.

6. The medical device probe of claim 4 wherein the illumination device is mounted on the elongate structures, the illumination device operatively connected by a fiber optic to the actuating mechanism.

7. The medical device probe of claim 2 wherein the illumination device is disposed on the catheter body proximate the tissue approximating structure, the illumination device connected by a conductor to the actuating mechanism.

8. The medical device probe of claim 2 wherein the illumination device is disposed on the catheter body proximate the tissue approximating structure, the illumination device connected by a fiber optic to the actuating mechanism.

9. The medical device probe of claim 1 wherein the illumination device is located within the inflatable balloon, said inflatable balloon comprising a translucent, transparent or light transmissive material.

10. The medical device probe of claim 9 wherein the inflatable balloon is filled with a fluorescent liquid to provide illumination, said inflatable balloon operatively connected to a supply lumen having an access port at the proximal end of the catheter.

11. The medical device probe of claim 1 wherein the illumination device is comprised of a light emitting diode or a fiber optic light.

12. The medical device probe of claim 1 wherein the illumination device is located caudate relative to the inflatable balloon, said inflatable balloon comprising a translucent, transparent or light transmissive material.

13. A method for performing a urethral anastomosis, the method comprising
inserting an illuminated anastomosis probe into the urethra, the anastomosis probe comprising an inflatable balloon, a tissue approximation structure and at least one illumination system,
activating the illumination system to position the inflatable balloon within the bladder,
inflating the balloon in the bladder, and
actuating the tissue approximation structure to hold the opposing ends of severed tissue in close proximity.

14. The method of claim 11 wherein the illumination system is located caudate relative to the inflatable balloon, said inflatable balloon comprising a translucent, transparent or light transmissive material.

15. The method of claim 14 wherein activating the illumination device further includes filling the inflatable balloon with a fluorescent liquid.

16. The method of claim 11 wherein the illumination system is located caudate relative to the inflatable balloon, said inflatable balloon comprising a translucent, transparent or light transmissive material.

17. The method of claim 11 wherein further includes activating the illumination system disposed proximate the tissue approximation structure.

18. The method of claim 17 wherein the tissue approximation structure includes at least one elongate member operably connected to the probe, the illumination system disposed on the elongate member.

19. The method of claim 17 wherein the tissue approximation structure includes at least one elongate member operably connected to the probe, the illumination system disposed on the perimeter of the probe adjacent to the elongate member.

20. A bladder neck illuminator device for visibly illuminating a bladder neck of a patient, comprising:
a hollow elongate flexible catheter body having a proximal and a distal end,
an inflatable balloon at the distal end,
a drainage lumen connected to the distal end,
first illumination means disposed adjacent to the inflatable balloon, the illumination means including an illumination lumen having a proximal end and a distal end disposed within the catheter body, an actuating mechanism proximate the distal end of the lumen and operatively connected to at least one illumination device, the illumination device disposed proximate the distal end of the illumination lumen;
tissue connector means disposed inboard of the inflatable balloon, said tissue connector means including a plurality of elongate members operably attached to the catheter body and activated by an actuation mechanism disposed within an actuation lumen within the catheter, and
second illumination means disposed adjacent to the elongate members, the second illumination means utilizing the illumination lumen disposed within the catheter body, the actuating mechanism for second illumination means disposed proximate the distal end of the lumen and operatively connected to at least one LED or fiber optic illumination device.
